(19) United States
(12) Patent Application Publication
(10) Pub. No.: US 2021/0299143 A1
(43) Pub. Date: Sep. 30, 2021

(54) USE OF ANTAGONISTS TO THE NUCLEAR STEROID RECEPTOR TO INHIBIT CORONAVIRUSES

(71) Applicant: Spectral Analytics, Inc., Las Vegas, NV (US)

(72) Inventors: Patrick T. Prendergast, New York, NY (US); Steven Adler, New York, NY (US)

(73) Assignee: Spectral Analytics, Inc., Las Vegas, NV (US)

(21) Appl. No.: 17/206,719

(22) Filed: Mar. 19, 2021

Related U.S. Application Data
(60) Provisional application No. 63/000,669, filed on Mar. 27, 2020.
(60) Provisional application No. 63/006,197, filed on Apr. 7, 2020.

(51) Int. Cl. A61K 31/567; A61K 31/4418; A61K 45/06

(52) U.S. Cl. CPC A61K 31/567; A61K 45/06; A61K 31/4418

(57) ABSTRACT

Compositions including at least one nuclear steroid family (NSF) receptor antagonist for the treatment or prevention of a coronavirus infection, such as SARS-CoV-2, and for the treatment of symptoms resulting from such infection. Also provided herein are methods of administering such compounds to a patient, either in a combination pharmaceutical formulation or in separate pharmaceutical formulations, to treat or prevent viral infections, including viral infections from coronaviruses such as SARS-CoV-2. Treatment decreases a viral load in a subject and/or may ameliorate symptoms associated with or produced by viral infection. Various administration modalities and mechanisms may be selected depending upon a condition of a patient and the patient's ability to be administered via a selected modality.

### CROSS REFERENCE TO RELATED APPLICATIONS

The present application claims priority to Provisional Patent Application U.S. 63/000,669 entitled USE OF ANTAGONISTS TO THE NUCLEAR STEROID RECEPTOR TO INHIBIT CORONAVIRUSES, filed on Mar. 27, 2020, and also to Provisional Patent Application U.S. 63/006,197 entitled USE OF ANTAGONISTS TO THE NUCLEAR STEROID RECEPTOR TO INHIBIT CORONAVIRUSES, filed on Apr. 7, 2020.

### BACKGROUND TO THE INVENTION

The nuclear steroid hormone receptor superfamily of ligand activated transcription factors is present in various tissues and is responsible for a broad range of effects in these tissues. This superfamily presently includes approximately 48 different proteins, most of which are understood to function as ligand activated transcription factors, exerting widely different biological responses by regulating gene expression. Members of this family include receptors for endogenous small, lipophilic molecules such as steroid hormones, retinoids, vitamin D, calcium channel blockers, and thyroid hormones. The nuclear steroid hormone superfamily includes steroid nuclear receptor subfamilies, including the mineralocorticoid receptor (MR; also referred to as the aldosterone receptor), the estrogen receptors (ER, including ER alpha and ER beta), the androgen receptor (AR), the progesterone receptors (PR), the glucocorticoid receptors (GR) and others. Also closely related in structure are the estrogen related receptors (ERRs), ERR-alpha, ERR-beta and ERR-gamma, and these are included within this superfamily. The nuclear steroid hormone superfamily performs many important functions in the body including regulating transcriptional homeostasis of electrolyte and water balance, growth, development and wound healing, fertility, stress responses, immunological function, and cognitive functioning. These effects may be mediated by cytosolic or nuclear events.

The nuclear steroid receptor family mediates its biological response by binding with ligands which cross the plasma membranes of cells and interact with ligand receptor proteins in the cytosol or nucleus to form complexes. The nuclear steroid receptor family forms complexes that may be chaperoned by heat shock proteins to the nucleus where they then accumulate in the nucleus of cells where they bind to specific regulatory DNA sequences. In publications, mineralocorticoid steroid receptors have been shown to be functionally present on lymphocytes (Leukemia (2000) 14, 1097-1104, Demonstration of the mineralocorticoid receptor hormone and action in human leukemic cell lines, N Mirshahi, S Mirshahi, N Golestaneh, Z Mishal, C Nicolas, C Hecquet and M K Agrwal).

Members of the steroid nuclear receptor subfamily exhibit significant homology to each other and possess closely related DNA and ligand binding domains. Given the very close similarity in ligand binding domains of the steroid nuclear receptors, it is not surprising that many naturally occurring and synthetic molecules possess the ability to modulate the activity of more than one steroid nuclear receptor.

Coronaviruses are related viruses that cause disease in mammals including humans. Coronaviruses include SARS-CoV, or SARS-CoV-1, which produces Severe Acute Respiratory Syndrome (SARS), MERS-CoV, which produces Middle East Respiratory Syndrome (MERS), and SARS-CoV-2, which produces the Coronavirus Disease 2019 (COVID-19). To date, no vaccine or antiviral drug has been shown to be sufficiently effective to treat or prevent coronavirus infections.

### SUMMARY OF THE INVENTION

The present invention is directed to compositions including at least one nuclear steroid family (NSF) receptor antagonist for the treatment or prevention of a coronavirus infection, such as SARS-CoV-2 and for the treatment of symptoms resulting from such infection. Additionally, additional NSF receptor antagonists may be included with the primary NSF receptor antagonist. The additional NSF receptor antagonists may be, for example, antagonists to a mineralocortoid/aldosterone receptor, antagonists to an estrogen receptor, antagonists to a glucocorticoid receptor, antagonists to an integrin receptor, antagonists to an androgen receptor, and/or calcium channel blockers. In particular aspects of the invention, the primary NSF receptor antagonist is a mineralocortoid antagonist and the additional NSF receptor antagonist is a calcium channel blocker. Such treatment may decrease the viral load in a subject and/or may ameliorate symptoms associated with or produced by the viral infection.

### DETAILED DESCRIPTION OF EMBODIMENTS OF THE INVENTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Except as otherwise indicated, standard methods known to those skilled in the art may be used for production of therapeutic agents, antibodies or antigen-binding fragments thereof, manipulation of nucleic acid sequences, production of transformed cells, modified proteins, packaging vectors, and transiently and stably transfected packaging cells. Such techniques are known to those skilled in the art. See, e.g., SAMBROOK et al., MOLECULAR CLONING: A LABORATORY MANUAL 2nd Ed. (Cold Spring Harbor, N.Y., 1989); F. M. AUSUBEL et al. CURRENT PROTOCOLS IN MOLECULAR BIOLOGY (Green Publishing Associates, Inc. and John Wiley & Sons, Inc., New York).

All publications, patent applications, patents, nucleotide sequences, amino acid sequences and other references mentioned herein are incorporated by reference in their entirety.

### General Definitions

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”). Moreover, the present invention also contemplates that in some embodiments of the invention, any feature or combination of features set forth herein can be excluded or omitted.

Furthermore, the term “about,” as used herein when referring to a measurable value such as an amount of a compound or agent of this invention, dose, time, temperature, and the like, is meant to encompass variations of ±10%, ±5%, ±1%, ±0.5%, or even ±0.1% of the specified amount.

The terms “comprise” and “comprising” and the terms “include” and “including” are used interchangeably in this specification and are to be afforded the widest interpretation.

As used herein, the transitional phrase “consisting essentially of” is to be interpreted as encompassing the recited materials or steps and those that do not materially affect the basic and novel characteristic(s) of the claimed invention. Thus, the term “consisting essentially of” as used herein should not be interpreted as equivalent to “comprising.”

The term “materially altered,” as applied to agents of the invention, refers to an increase or decrease in biological activity of at least about 50% or more as compared to the activity of an agent described.

As used herein, the term “amino acid” encompasses any naturally occurring amino acids, derivatives thereof, and synthetic amino acids, including non-naturally occurring amino acids. Further, the non-naturally occurring amino acid can be an “unnatural” amino acid as described by Wang et al., (2006) Annu. Rev. Biophys. Biomol. Struct. 35:225-49.

Naturally occurring, levorotatory (L-) amino acids are shown in Table 1.

TABLE 1

Abbreviation

| | Three-Letter | One-Letter |
|---|---|---|
| Amino Acid Residue | Code | Code |
| Alanine | Ala | A |
| Arginine | Arg | R |
| Asparagine | Asn | N |
| Aspartic acid | Asp | D |
| (Aspartate) | | |
| Cysteine | Cys | C |
| Glutamine | Gln | Q |
| Glutamic acid | Glu | E |
| (Glutamate) | | |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

A “functional fragment” of an agent, as used herein, means a portion of a larger agent that substantially retains its ability to perform its therapeutic function, such as reduce inflammation, reduce C-reactive protein, and/or reduce inflammatory cytokines.

As used herein, the term “derivative” is used to refer to an agent which differs from a naturally occurring protein or a functional fragment by minor modifications to the naturally occurring agent, but which substantially retains the biological activity of the naturally occurring protein. Minor modifications include, without limitation, changes in one or a few amino acid side chains, changes to one or a few amino acids (including deletions, insertions, and/or substitutions), changes in stereochemistry of one or a few atoms (e.g., D-amino acids), and minor derivatizations, including, without limitation, methylation, glycosylation, phosphorylation, acetylation, myristoylation, prenylation, palmitation, amidation, and addition of glycosylphosphatidyl inositol.

By “substantially retain” a property, it is meant that at least about 50%, 60%, 70%, 75%, 85%, 90%, 95%, 97%, 98%, 99% or 100% of the property (e.g., activity or other measurable characteristic) is retained.

The term “antagonist” refers to any compound that binds, interacts, or otherwise blocks a biological receptor so as to block or decrease a biological response by that receptor. The definition includes full antagonists as well as partial antagonists, whereby a “partial antagonist” should be understood as a compound capable of partially, but not fully, in-activating a receptor.

The term “steroid” refers to lipids that contain a hydrogenated cyclopentanoperhydrophenanthrene ring system.

By the term “treat,” “treating,” or “treatment of” (or grammatically equivalent terms) is meant to reduce or to at least partially improve or ameliorate the severity of the subject's condition and/or to alleviate, mitigate or decrease in at least one clinical symptom and/or to delay the progression of the condition. As such, “treat” includes both inhibiting viral replication and decreasing viral load, and also includes treating symptoms associated with the viral infection.

As used herein, the term “prevent,” “prevents,” or “prevention” (and grammatical equivalents thereof) means to delay or inhibit the onset of a disease. The terms are not meant to require complete abolition of disease and encompass any type of prophylactic treatment to reduce the incidence of the condition or delays the onset of the condition.

The term “therapeutically effective amount” or “effective amount,” as used herein, refers to that amount of a composition, compound, or agent of this invention that imparts a modulating effect, which, for example, can be a beneficial effect, to a subject afflicted with a disorder, disease or illness, including improvement in the condition of the subject (e.g., in one or more symptoms), delay or reduction in the progression of the condition, prevention or delay of the onset of the disorder, and/or change in clinical parameters, disease or illness, etc., as would be well known in the art. For example, a therapeutically effective amount or effective amount can refer to the amount of a composition, compound, or agent that improves a condition in a subject by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

“Pharmaceutically acceptable,” as used herein, means a material that is not biologically or otherwise undesirable, i.e., the material can be administered to an individual along with the compositions of this invention, without causing substantial deleterious biological effects or interacting in a deleterious manner with any of the other components of the composition in which it is contained. The material would naturally be selected to minimize any degradation of the active ingredient and to minimize any adverse side effects in the subject, as would be well known to one of skill in the art (see, e.g., Remington's Pharmaceutical Science; 21^{st} ed. 2005).

“Concurrently” means sufficiently close in time to produce a combined effect (that is, concurrently can be simultaneously, or it can be two or more events occurring within a short time period before or after each other). In some embodiments, the administration of two or more compounds “concurrently” means that the two compounds are administered closely enough in time that the presence of one alters the biological effects of the other. The two compounds can be administered in the same or different formulations or sequentially. Concurrent administration can be carried out by mixing the compounds prior to administration, or by administering the compounds in two different formulations, for example, at the same point in time but at different anatomic sites or using different routes of administration.

### Active Compounds and Compositions

The present inventor has discovered that certain sequences present on coronaviruses can interfere and bind to the lipophilic regions associated with the nuclear steroid hormone family (NSF) receptor system and inhibit the action of protein receptors (e.g., ACE-2) that can modulate endothelial permeability, endothelial nitric oxide and reactive oxygen species and the actin cytoskeleton. This may create over exposure to aldosterone in the endothelial of the cell, which may cause the production of inflammatory cytokines and nitric oxide and may stimulate an inflammatory cascade that may particularly affect the patient's lungs. Thus, compounds that antagonize the NSF receptor system, e.g., the mineralocortoid receptor, and inhibit the activity of aldosterone in the body may inhibit coronavirus replication and side effects resultant from the coronavirus infection.

Specifically, the outer coat of a lipid envelope virus is constructed of several types of polypeptide chains often arranged in several layers. In many viruses, the protein capsid is further enclosed by a lipid bilayer membrane that contains proteins. Many of these enveloped proteins acquire this envelope in the process of budding from the plasma membrane. This budding process allows the virus particles to leave the cell without disrupting the plasma membrane and therefore not killing the cell. However, the coat protein of the lipid envelope virus is significantly different in phospholipid profile than that of the plasma membranes of the host cell. Without being bound by any theory, it is thought that this is due to selective sequestration of lipids occurring through the budding process, in which the coronavirus viral proteins select specific domains within the host cell membrane through which to emerge during maturation. Aldosterone is one hormone that is selected and sequestered during the budding process of the coronavirus. A high cholesterol/phospholipid ratio within viral envelopes may increase the infectivity of a coronavirus.

The advantage of administering a pharmaceutical formulation containing at least one antagonist of the nuclear steroid receptor family for the treatment of coronavirus is twofold: (1) the antagonist of a NSF receptor may inhibit or prevent coronavirus replication and eliminate the virus's ability to cause vascular endothelial instability; and (2) the antagonist of the NSF receptor may have immune upregulatory properties, for example in macrophages and/or dendritic cells, whereby inflammatory cytokines, nitrites, and reactive oxygen species are inhibited and immune cells are prevented from undergoing apoptosis from exposure to coronavirus coat proteins.

The present inventor has also discovered that calcium channel blockers may also work synergistically with the NSF receptor antagonists to undermine the pathogenicity of coronaviruses and stabilize vascular endothelial cells, which may, in some cases, result in a broad-spectrum inhibition of viral stimulated inflammatory symptoms. Furthermore, it has been found that certain therapeutic agents administered with the NSF receptor antagonist and/or the calcium channel blockers may also provide additional therapeutic effects in the treatment of coronavirus infections.

Thus, the present invention is directed to compositions including at least one nuclear NSF receptor antagonist for the treatment of a coronavirus infection and symptoms resulting therefrom. Additionally, additional NSF receptor antagonists may be included with the primary NSF receptor antagonist. The additional NSF receptor antagonists may be, for example, antagonists to the mineralocortoid/aldosterone receptor, antagonists to the estrogen receptor, antagonists to the glucocorticoid receptor, antagonists to the integrin receptor, antagonists to the androgen receptor, and/or calcium channel blockers. In particular aspects of the invention, the primary NSF receptor antagonist is a mineralocortoid antagonist and the additional NSF receptor antagonist is a calcium channel blocker.

In addition, such compositions may further include a therapeutic agent that is targeted to reduce inflammation, such as by reducing the amount of C-reactive protein and/or inflammatory cytokines. Also provided herein are methods of using such active compounds, either in a combination formulation or in separate pharmaceutical formulations, to treat or prevent viral infections, including viral infections from coronaviruses such as SARS-CoV-2. Such treatment may decrease the viral load in a subject and/or may ameliorate symptoms associated with or produced by the viral infection.

NSF receptor antagonists include any compound that is an antagonist to one or more of receptors in the nuclear steroid family, including, without limitation, a (1) mineralocorticoid/aldosterone receptor; (2) estrogen receptor; (3) progesterone receptor; (4) glucocorticoid receptor; (5) integrin receptor; (6) androgen receptor; and/or (7) calcium channel receptors. Any suitable NSF receptor antagonist may be included in compositions and method of the invention. However, in particular embodiments, the NSF receptor antagonist is a mineralocortoid antagonist.

In some embodiments of the invention, the mineralocortoid antagonist includes one or more progesterones having mineralocortoid activity. Examples of progesterones include, without limitation, progesterone, gestodene, drospirenone, dimethisterone, ethinyl estradiol, ethisterone, 11β-hydroxyprogesterone, 17α-hydroxyprogesterone, 16α-methyl progesterone, hydroxyprogesterone caproate, medroxyprogesterone acetate, proligestone, metapristone, and mifepristone (11β-[p-(Dimethylamino)phenyl]-17β-hydroxy-17-(1-propynyl)estra-4,9-dien-3-one). In some embodiments, such compounds are halogenated, for example, at the 9α position. Exemplary halogens include chlorine, bromine, fluorine and iodine.

In some embodiments, the antagonist of the mineralocortoid receptor includes a 7α-acetylthio-4-pregnene-3,20-dione compound having Formula B:

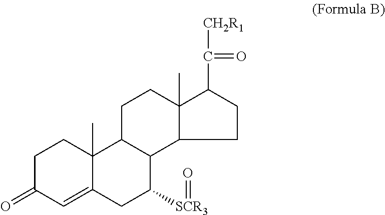

wherein R₁ is hydrogen, hydroxyl, a mineral acid ester such as a sulfate, phosphate or nitrate group, or acyloxy-OR₂, the acyl group R₂ being derived from a carboxylic acid of the formula R₄OOH which may have up to 12 carbon atoms, and in which R₄ may be substituted or unsubstituted, saturated or unsaturated, straight chain or branched, alicyclic, aryl, heterocyclic or mixed group, and R₃ is methyl. In one embodiment, R₁ is hydroxyl or OR₂ where R₂ is defined as above, wherein in particular embodiments, R₂ has 3 to 12 carbon atoms. In another embodiment R₁ is hydroxy or a monocarboxylic, straight or branched chain alkanooyloxy group having up to 12 carbon atoms. In another embodiment R₁ is hydrogen, hydroxy, acetoxy, propionyloxy, n-butyryloxy, trimethylacetoxy, n-valeroyloxy or n-heptanoyloxy. In some embodiments, such compounds are halogenated, for example, at the 9α position. Exemplary halogens include chlorine, bromine, fluorine and iodine.

In some embodiments of the invention, the antimineralocortoid antagonist includes one or more of 7α-acetylthio-4-pregnene-3,20-dione; 7α-acetylthio-21-hydroxy-4pregnene-3,20-dione; 7α-acetylthio-21-acetoxy-4-pregnene-3,20-dione; 7α-acetylthio-21-propionyloxy-4-pregnene-3,20-dione; 7α-acetylthio-21-n-butyryloxy-4 pregnene-3,20-dione; 7α-acetylthio-21-trimethylacetoxy-4-pregnene-3,20-dione; 7α-acetylthio-21-n-valeroyloxy-4-pregnene-3,20-dione; and 7α-acetylthio-21-heptanoyloxy-4-pregnene-3,20-dione. In some embodiments, such compounds are halogenated, for example, at the 9a position. Exemplary halogens include chlorine, bromine, fluorine and iodine.

In particular embodiments of the invention, the mineralocorticoid antagonist includes mifepristone.

In some embodiments of the invention, the mineralocortoid antagonist includes a spirolactone. Examples of spirolactones include, without limitation, spironolactone, spirorenone, dihydrospirenone, 1,2-dihydro-spirorenone, 1,2α-methylene-spirorenone, 7α-Acetylthio-3-oxo-4,15-androstadiene-[17(β-1′)-spiro-5′]perhydrofuran-2′-one, 3-Oxo-7α-propionylthio-4,15-androstadiene-[17(β-1′)-spiro-5′]perhydrofuran-2′-one, 6β,7β-methylene-3-oxo-4,15-androstadiene-[17(β-1′)-spiro-5′]perhydrofuran-2′-one, 15α,16α-methylene-3-oxo-7α-propionylthio-4-androstene-[17(β-1′)-spiro-5′]perhydrofuran-2′-one, 6β,7β,15α,16α-dimethylene-3-oxo-4-androstene-[17(β-1′)-spiro-5′]perhydrofuran-2′-one, 7α-acetyithio-15α,16α-methylene-3-oxo-4-androstene-[17(β-1′)-spiro-5′]perhydrofuran-2′-one, 7α-acetylthio-15β,16β-methylene-3-oxo-4-androstene-[17(β-1′)-spiro-5′]perhydrofuran-2′-one, 15β,16β-methylene-3-oxo-7β-propionylthio-4-androstene-[17(β-1′)-spiro-5′]perhydrofuran-2′-one, 6β,7β,15β,16β-Dimethylene-3-oxo-4-androstene-[17(β-1′)-spiro-5′]perhydrofuran-2′-one, eplerenone, iseplerenone, potassium canrenoate, canrenoate, canrenone, 7-α-thiospironolactone, 7α-thiomethylspironolactone, 6-β-hydroxy-7-α-thiospironolactone, and 6-β-hydroxy-7-α-thiomethylspironolactone. Additional spirolactones can be found, for example, in U.S. Pat. Nos. 4,129,564, 4,529,811, and 5,981,744, and in WO 2000/033847, which are all incorporated herein by reference in their entirety. In some embodiments, such compounds are halogenated, for example, at the 9a position. Exemplary halogens include chlorine, bromine, fluorine and iodine.

Integrin receptor antagonists may also be used. Examples of integrin receptor antagonists that may be used herein may be found in U.S. Pat. No. 9,896,480, which is herein incorporated by reference in its entirety. In some embodiments, such compounds are halogenated, for example, at the 9α position. Exemplary halogens include chlorine, bromine, fluorine and iodine.

In another embodiment of the invention, the antagonist of the mineralocortoid receptor is a 9,11-epoxy steroid compound, and in particular embodiments, those 9,11-epoxy steroid compounds of the 20-spiroxane series and their analogs may be used. In some embodiments, such compounds are halogenated, for example, at the 9a position. Exemplary halogens include chlorine, bromine, fluorine and iodine.

Other NSF receptor antagonists that may be used in embodiments of the invention include aglepristone, cyproterone, cyproterone acetate, casodex, besylate, bicalutamide, clomifene, femarelle, ormeloxifene, raloxifene, tamoxifen, toremifene, lasofoxifene, and ospemifene, nimodipine, afimoxifene, arzoxifene, fulvestrant, bazedoxifene, flutamide, nilutamide.

In addition, in some embodiments of the invention, the NSF receptor antagonist may include an idiotypic humanized monoclonal antibody to the mineralocortoid binding site and/or an anti-idiotypic humanized monoclonal antibody to the estrogen receptor site. The term idiotypic antibody is an antibody raised against the antigen binding site of another antibody. The antibody may be produced using an anti-idiotypic method. For example, a monoclonal idiotypic antibody that is an anti-aldosterone antibody is raised against the aldosterone binding site or any other lipophilic ligand binding site on the steroid nuclear receptor.

In some embodiments of the invention, compositions include at least one calcium channel blocker. Any suitable calcium channel blocker may be used, but in some embodiments, the calcium channel blocker is also an NSF receptor antagonist. Examples of calcium channel blockers that may be used in some embodiments of the invention include, without limitation, amlodipine, aranidipine, azelnidipine, barnidipine, benidipine, cilnidipine, clevidipine, efonidipine, felodipine, isradipine, lacidipine, lercanidipine, manidipine, nicardipine, nifedipine, nilvadipine, nimodipine, nisoldipine, nitrendipine, and pranidipine.

For each of the NSF receptor antagonists, calcium channel blockers, any pharmaceutically acceptable salts, polymorphs, hydrates, clathrates, solvates, inclusion compounds, isomers, complexes, metabolites, and prodrugs (collectively referred to herein as “pharmaceutically acceptable salts, metabolites, and prodrugs”) may be used in embodiments described herein. Examples of salts include without limitation, acetate, adipate, alginate, anthranilate. aspartate, benzoate, butyrate, cinnamate, citrate, formate, fumarate, glycolate, hemisulfate, heptanoate, hexanoate, hydrochloride, hydrobromide, hydroiodide, 2-hydroxyethanesulfonate, lactate, maleate, malonate, methanesulfonate, naphthalene sulfonate, nicotinate, nitrate, oxalate, perchlorate, palmoate, pectinate, persulfate, phosphate, hydroxynapthoate, pivalate, propionate, pyruvate, salicylate, succinate, sulfanilate, sulfate, tartrate, thiocyanate, tosylate and undecanoate. Other suitable pharmaceutically acceptable salts are known in the art. Furthermore, one of skill in the art will understand which salts may be appropriate for the particular compounds described herein.

The term “prodrug” refers to compounds that are transformed in vivo to yield the parent compound of the above formula, for example, by hydrolysis in blood, see, e.g., T. Higuchi and V. Stella, Prodrugs as Novel delivery Systems, Vol. 14 of the A.C.S. Symposium Series and in Edward B. Roche, ed., Bioreversible Carriers in Drug Design, American Pharmaceutical Association and Pergamon Press, 1987, both of which are incorporated by reference herein. See also U.S. Pat. No. 6,680,299. Exemplary prodrugs include a prodrug that is metabolized in vivo by a subject to an active drug having an activity of the compounds as described herein, wherein the prodrug is an ester of an alcohol or carboxylic acid group, if such a group is present in the compound; an amide of an amine group or carboxylic acid group, if such groups are present in the compound; a urethane of an amine group, if such a group is present in the compound; an acetal or ketal of an alcohol group, if such a group is present in the compound; a N-Mannich base or an imine of an amine group, if such a group is present in the compound; or a Schiff base, oxime, acetal, enol ester, oxazolidine, or thiazolidine of a carbonyl group, if such a group is present in the compound, such as described, for example, in U.S. Pat. Nos. 6,680,324 and 6,680,322. Methods of masking charged or reactive moieties as a pro-drug are known by those skilled in the art (see, e.g., P. Korgsgaard-Larsen and H. Bundgaard, A Textbook of Drug Design and Development, Reading U.K., Harwood Academic Publishers, 1991).

As used herein, such prodrugs are pharmaceutically acceptable prodrug. The term “pharmaceutically acceptable prodrug” (and like terms) as used herein refers to those prodrugs of compounds of the invention which are, within the scope of sound medical judgment, suitable for use in contact with the tissues of humans and/or other animals without undue toxicity, irritation, allergic response and the like, commensurate with a reasonable risk/benefit ratio, and effective for their intended use, as well as the zwitterionic forms, where possible, of the compound of the invention.

The NSF receptor antagonists and calcium channel blockers described above may be obtained or synthesized by any method known in the art.

Another aspect of the invention relates to pharmaceutical formulations including the active compounds that are suitable for use in the methods of the invention. One or more of the NSF receptor antagonist, the calcium channel blocker, and/or the therapeutic agent (also referred to herein as the “active compounds”) may be combined into a pharmaceutically acceptable composition. Alternatively, each active compound may be prepared and administered in a separate pharmaceutical formulation. In some embodiments, the composition is a pharmaceutical composition comprising one or more active compounds and a pharmaceutically acceptable carrier. As used herein, the term “active compounds” includes all pharmaceutically acceptable salts, metabolites, and prodrugs.

In particular embodiments of the present invention, at least one of the active compounds is micronized. In accordance with the present invention, the expression “micronized” means that the compound has been micronized in accordance with any process for micronizing, several which are known in the art. In some embodiments, the micronized particles have an average diameter of about 10 microns or less, and in some cases, about 5 microns or less. For example, in a particular aspect of the invention, at least 80% of the particles in a formulation of micronized particles have a diameter of less than 5 microns. In some embodiments, only some of the active compounds are micronized. For example, in some cases, only the NSF receptor antagonist and/or the calcium channel blocker is micronized.

Any suitable pharmaceutical formulation or dosage form may be used to administer the active compounds. In some embodiments, one or more of the active compounds are in a liquid form suitable for intravenous administration. In some embodiments, one or more of the active compounds are provided in a form suitable for oral administration. In yet further embodiments, one or more active compounds are present in a liquid preparation suitable for intramuscular or subcutaneous injection. Such forms include, without limitation, tablets, capsules, dragees, liquid preparations, troches, lozenges, melts, powders, micronized particles, non-micronized particles, solutions, emulsions, elixirs, suspensions, syrups or inhalations and controlled release forms thereof. In some embodiments, all of the active compounds are present in one dosage form, while in other embodiments, the active compounds are formulated and administered in separate dosage forms. In the discussion below, whether indicated specifically or not, one of skill in the art will appreciate any dosage form described with respect to a single active compound could be used in a combination formulation.

In some embodiments of the invention, an active compound of the invention is provided in a tablet or capsule dosage form, either in an immediate release or extended release formulation. In some embodiments, the dosage form is an immediate release tablet that releases at least 85%, e.g., at least 85%, 90%, 95%, 96%, 97%, 98%, or 99%, of the active compounds contained therein within a period of less than 15 minutes after administration of the tablet to a subject. Formulations of the active compounds, including immediate release or extended release formulations, may be processed into unit dosage forms suitable for oral administration, such as for example, filled capsules, compressed tablets or caplets, or other dosage form suitable for oral administration using conventional techniques. Immediate release dosage forms prepared as described may be adapted for oral administration, so as to attain and maintain a therapeutic level of the compounds over a preselected interval. In certain embodiments, an immediate release dosage form as described herein may comprise a solid oral dosage form of any desired shape and size including round, oval, oblong cylindrical, or polygonal.

In particular, when the immediate release formulations are prepared as a tablet, the immediate release tablets may contain a relatively large percentage and absolute amount of the compounds and so are expected to improve patient compliance and convenience, by replacing the need to ingest large amounts of liquids or liquid/solid suspensions. One or more immediate release tablets as described herein can be administered, by oral ingestion, e.g., closely spaced, in order to provide a therapeutically effective dose of the compound to the subject in a relatively short period of time.

Where desired or necessary, the outer surface of an immediate release dosage form may be coated, e.g., with a color coat or with a moisture barrier layer using materials and methods known in the art. For example, where the active compounds delivered by the unit dosage form is highly hygroscopic, providing a moisture barrier layer over the immediate release dosage form as disclosed herein may be desirable. Protection of an immediate release dosage form as disclosed herein from water during storage may be provided or enhanced by coating the tablet with a coating of a substantially water soluble or insoluble polymer. Useful water-insoluble or water-resistant coating polymers include ethyl cellulose and polyvinyl acetates. Further water-insoluble or water-resistant coating polymers include polyacrylates, polymethacrylates or the like. Suitable water-soluble polymers include polyvinyl alcohol and HPMC. Further suitable water-soluble polymers include PVP, HPC, HPEC, PEG, HEC and the like.

Enteric coatings may also be used. In some embodiments of the invention, the enteric coating is made of a polymer, such as, for example, poly(lactic-acid) polyester, cellulose acetate phthalate, hydroxypropyl-methyl cellulose phthalate poly(butyl methacrylate), (2-dimethyl aminoethyl) methacrylate, and methyl methacrylate.

According to a further aspect of the invention, the compounds are formulated in a liposome or a hydrophilic molecular cage, such as a cyclodextrin cage. A liposome or cyclodextrin cage may carry the active compounds to coronavirus infected cells as the liposome may be modified to target to the coronavirus specifically, such as by including antibodies to coronavirus spike proteins on the liposomes. The advantage of this is that the liposome can selectively target viral infected cells.

The compounds of the invention, or pharmaceutically acceptable salts, derivatives, or prodrugs thereof, can be formulated and administered as free bases or in the form of their pharmaceutically acceptable salts for purposes of stability, convenience of crystallization, increased solubility, and the like.

In certain embodiments, for each dosage form, the active compound(s) are in total present in an amount of about 90%, 90.5%, 91%, 91.5%, 92%, 92.5%, 93%, 93.5%, 94%, 94.5%, 95%, 95.5%, 96%, 96.5%, 97%, 97.5%, or 98% by weight of the tablet or any value or range therein. In certain embodiments, the active compounds are in total present in an amount of about 90% to about 98%, about 92% to about 98%, about 94% to about 98%, about 96% to about 98%, about 90% to about 92%, about 90% to about 94%, about 90% to about 96%, about 92% to about 94%, about 92% to about 96%, or about 94% to about 96%.

In certain embodiments, each dosage form includes at least one binder present in an amount of about 1%, 1.5%, 2%, 2.5%, 3%, 3.5%, 4%, 4.5%, or 5% by weight of the tablet or any value or range therein. In certain embodiments, the at least one binder is present in an amount of about 1% to about 5%, about 2% to about 5%, about 3% to about 5%, about 4% to about 5%, about 1% to about 2%, about 1% to about 3%, about 1% to about 4%, about 2% to about 3%, about 2% to about 4%, or about 3% to about 4%. The tablet may comprise at least one binder, e.g., 1, 2, 3, 4, 5, or more binders.

In certain embodiments, the at least one binder is selected from at least one of hydroxypropyl cellulose, ethylcellulose, hydroxypropyl methylcellulose, polyvinyl alcohol, hydroxyethyl cellulose, povidone, copovidone, pregelatinized starch, dextrin, gelatin, maltodextrin, starch, zein, acacia, alginic acid, carbomers (cross-linked polyacrylates), polymethacrylates, sodium carboxymethylcellulose, guar gum, hydrogenated vegetable oil (type 1), methylcellulose, magnesium aluminum silicate, and sodium alginate or any combination thereof. In some embodiments, the at least one binder is hydroxypropyl cellulose. In another embodiment the composition further includes a pharmaceutically acceptable carrier, which in one embodiment is cyclodextrin, preferably hydroxypropyl beta cyclodextrin.

In certain embodiments, each dosage form includes at least one lubricant in an amount of about 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, or 2.0% by weight of the tablet or any value or range therein. In certain embodiments, the at least one lubricant is present in an amount of about 0.1% to about 2.0%, about 0.5% to about 2.0%, about 1.0% to about 2.0%, about 1.5% to about 2.0%, about 0.1% to about 0.5%, about 0.1% to about 1.0%, about 0.1% to about 1.5%, about 0.5% to about 1.0%, about 0.5% to about 1.5%, or about 1.0% to about 1.5%. The tablet may comprise at least one lubricant, e.g., 1, 2, 3, 4, 5, or more lubricants. Where the immediate release formulation is provided as a tableted dosage form, still lower lubricant levels may be achieved with use of a “puffer” system during tableting. Such systems are known in the art, commercially available and apply lubricant directly to the punch and die surfaces rather than throughout the formulation.

In certain embodiments, the at least one lubricant is selected from at least one of magnesium stearate, stearic acid, calcium stearate, hydrogenated castor oil, hydrogenated vegetable oil, light mineral oil, magnesium stearate, mineral oil, polyethylene glycol, sodium benzoate, sodium stearyl fumarate, and zinc stearate or any combination thereof. In some embodiments, the at least one lubricant is magnesium stearate. In other embodiments, magnesium stearate may be used in combination with one or more other lubricants or a surfactant, such as sodium lauryl sulfate. In particular, if needed to overcome potential hydrophobic properties of magnesium stearate, sodium lauryl sulfate may also be included when using magnesium stearate (Remington: the Science and Practice of Pharmacy, 20^{th} edition, Gennaro, Ed., Lippincott Williams & Wilkins (2000)).

Suitable injectable solutions include intravenous, subcutaneous and intramuscular injectable solutions. Examples of injectable forms include solutions, suspensions and emulsions. Typically the compound(s) is injected in association with a pharmaceutical carrier such as normal saline, Ringers solution, dextrose solution and other aqueous carriers known in the art. Appropriate non-aqueous carriers may also be used and examples include cyclodextrin, preferably hydroxypropyl beta cyclodextrin, mixed oils (vitamin E oil), polyethylene glycol and ethyl oleate. A preferred carrier is cyclodextrin in water. It may be desirable to include additives in the carrier such as buffers and preservatives or other substances to enhance isotonicity and chemical stability. The compositions may also be administered in the form of an implant.

The concentration of each active agent in a particular dosage form may vary based on the dosage form, the number of doses administered, and the method of administration used. Thus, any dosage form that provides a therapeutically effective amount of an active compound of the invention may bs used.

In some embodiments of the invention, the NSF antagonist dosage form is a tablet that contains about 1 mg to about 900 mg of the NSF receptor antagonist, or any range or value therein, e.g., about 100 mg to about 500 mg, or e.g., about 100 mg to about 300 mg. In particular embodiments, the NSF receptor antagonist is mifepristone and the mifepristone is present in a tablet or capsule dosage form at a concentration in a range of about 100 mg to about 300 mg (e.g., 200 mg).

In some embodiments of the invention, the calcium channel blocker dosage form is a tablet that contains a calcium channel blocker at a concentration in a range of 0.1 to about 1000 mg, and any range or value therein, e.g., in a range of about 1 to about 100 mg, e.g., about 1 to about 5 mg, e.g., about 1 to 3 mg. In particular embodiments, the calcium channel blocker is amlodipine, which is present in a tablet or capsule dosage form at a concentration in range of about 1 to about 5 mg (e.g., 2.5 mg).

The dosage forms disclosed herein can also be provided as a kit comprising, separately packaged, a plurality of active compound tablets or other preparations. Such tablets or other preparations may be combination compositions that include all active compounds, or there may be separate tablets/liquid preparation for each active compound. Such tablets/preparations can be individually packaged, such as in foil envelopes or in a blister pack. The tablets can be packaged in many conformations with or without desiccants or other materials to prevent ingress of water. Instruction materials or means, such as printed labeling, can also be included for their administration, e.g., sequentially over a preselected time period and/or at preselected intervals, to yield the desired levels of active compounds in vivo for preselected periods of time, to treat a preselected condition.

Provided according to particular embodiments of the invention are kits that include a plurality of dosage forms that include a NSF receptor antagonist and a calcium channel blocker. Such dosage forms may be capsules or tablets, or in some cases, may be liquid preparations for IV administration or injection. Such a kit may include the number of doses so as to effectively treat or prevent the coronavirus infection, or such a kit may include the number of doses for a particular time period, such as 24, 48, 72, or 96 hours.

Provided according to other embodiments of the invention are kits that include separate dosage forms for each of the active compounds. For example, the kit may include a plurality of tablets, capsules, or other preparations of the NSF receptor antagonist; a plurality of tablets, capsules, or other preparations of the calcium channel blocker, and a plurality of tablets, capsules, or other preparations. Such a kit may sequester a dose of each active compound together so as to indicate the compounds to administer at a specific time point. Such a kit may include the number of doses so as to effectively treat or prevent the coronavirus infection, or such a kit may include the number of doses for a particular time period, such as 24, 48, 72, or 96 hours.

In particular embodiments of the invention, the kit includes a plurality of tablets or capsules of mifepristone as the NSF receptor antagonist, a plurality of tablets or capsules of amlodipine as the calcium channel blocker, and liquid preparation for intramuscular or subcutaneous injection of a therapeutic agent. In such a kit, the doses for each active compound to be administered at a particular time point may be sequestered so as to indicate that such doses should be administered concurrently or sequentially. In other embodiments, the kits include a plurality a liquid preparations of mifepristone, and a plurality of liquid preparations of amlodipine.

### Methods of Treatment and Prevention of Coronavirus Infection

The present invention further contemplates administering to a subject an amount of at least one NSF receptor antagonist, and optionally a calcium channel blocker and/or a therapeutic agent, for the treatment or prevention of a coronavirus infection. Such compositions may be therapeutically effective to inhibit coronavirus replication, may lower the circulating viral load, and may treat or prevent vascular endothelial instability, muscle pain, and rheumatic pain which the subject may suffer from coronavirus infection. Treating as used herein may also include treating other symptoms of such viral infections.

The methods and compositions described herein may be used to treat any coronavirus in the family of Coronaviridae. Examples of coronaviruses include SARS-CoV, or SARS-CoV-1, which produce Severe Acute Respiratory Syndrome (SARS), MERS-CoV, which produces Middle East Respiratory Syndrome (MERS), and SARS-CoV-2, which produces the Coronavirus Disease 2019 (COVID-19). In particular embodiments of the invention, the methods and compositions are used to treat or prevent the viral infection by SARS-CoV-2 and/or treat the symptoms produced by COVID-19. As used herein, treating COVID-19 refers to both treatment of SARS-CoV-2 virus, or any mutated or related viral strain thereof, and treating the symptoms of COVID-19. As used herein, the term “SARS-CoV-2” includes both the known SARS-CoV-2 viral strains, and also any related strains, including those created by viral mutation.

The present invention finds use in research as well as veterinary and medical applications. Suitable subjects are generally mammalian subjects. The term “mammal” as used herein includes, but is not limited to, humans, non-human primates, cattle, sheep, goats, pigs, horses, cats, dog, rabbits, rodents (e.g., rats or mice), etc. Human subjects include neonates, infants, juveniles, adults and geriatric subjects. In particular embodiments, the subject is a human subject that has been infected with a coronavirus such as SARS-CoV-2 or may be exposed to such a coronavirus.

In some embodiments of the invention, one or more compositions according embodiments of the invention is administered to a subject to treat or prevent coronavirus infection. The subject can be a subject “in need of” the methods of the present invention, e.g., in need of the therapeutic effects or benefits of the inventive methods. For example, the subject can be a subject that has been tested and confirmed to have been infected with a coronavirus; a subject that is experiencing symptoms consistent with coronavirus; a subject that is suspected of having infection by a coronavirus; and/or is a subject that is anticipated to become exposed to a coronavirus, such that the methods and compositions of the invention are used for therapeutic and/or prophylactic treatment.

In one embodiment of the invention, the compositions according to embodiment of the invention are administered to a subject in need thereof to treat or prevent a coronavirus infection. The active compounds can be administered continuously or intermittently. In one embodiment, the active compounds are administered to the subject more than once a day, e.g., 2, 3, or 4 times per day, or once every 1, 2, 3, 4, 5, 6, or 7 days. In another embodiment, the active compounds are administered to the subject no more than once a week, e.g., no more than once every two weeks, once a month, once every two months, once every three months, once every four months, once every five months, once every six months, or longer. In a further embodiment, the active compounds are administered using two or more different schedules, e.g., more frequently initially (for example to build up to a certain level, e.g., once a day or more) and then less frequently (e.g., once a week or less). In other embodiments, the active compounds can be administered by any discontinuous administration regimen. The administration can continue for one, two, three, or four weeks or one, two, or three months, or longer. Optionally, after a period of rest, the compound can be administered under the same or a different schedule. The period of rest can be one, two, three, or four weeks, or longer, according to the pharmacodynamic effects of the compound on the subject. In another embodiment the compound can be administered to build up to a certain level, then maintained at a constant level and then a tailing dosage. Each of the active compounds may be administered concurrently or sequentially, or in some cases, each of the active compounds may be administered on a separate dosage schedule discussed above.

In one aspect of the invention, one or more of the active compounds are delivered to a subject concurrently or sequentially with an additional therapeutic agent. The additional therapeutic agent can be delivered in the same composition as the compound or in a separate composition. The additional therapeutic agent can be delivered to the subject on the same or different schedule as the active compounds and by the same or different route as compared to the active compounds. The additional therapeutic agent can be any agent that provides a benefit to the subject.

In particular embodiments, the additional therapeutic agent is an antiviral agent. Examples of antiviral agents that may be administered concurrently or sequentially with the compositions of the invention include, without limitation, Carbocyclic 3-deazaadenosine (C-c³Ado), R- and S-isomers of 6′-C-neplanocin A analogues, carbocyclic analogues of adenosine, aristeromycin (carbocyclic adenosine), carbocyclic 3-deazaadenosine, neplanocin A (NepA), 3-deazaneplanocin A, 5′-nor derivatives of aristeromycin, carbocylic 3-deazaadenosine, 2-halo (e.g., 2-fluoro) and 6′-R-alkyl (e.g., 6′-R-methyl) derivatives neplanocin A, 9-(hydroxyalkenyl)purines (adenines and 3-deazaadenines), which analogues of neplanocin A, 3-deazaneplanocin A, bromine epiandrosterone, the 5′-nor derivatives of carbocyclic adenosine (C-Ado, aristeromycin), the 2-halo (i.e., 2-fluoro) and 6′-R-alkyl (i.e., 6′-R-methyl) derivatives of neplanocin A, 6′-C-methylneplanocin A (isomers I and II), 5′-noraristeromycin, (S)-9-(2,3-dihydroxypropyl)adenine, 5′-nor derivatives of carbocyclic adenosine (C-Ado, aristeromycin), 2-halo (i.e., 2-fluoro) and 6′-R-alkyl (e.g., 6′-R-methyl) derivatives of neplanocin A, 9-(hydroxyalkyl)-3-deazaadenines, which are analogues of the carbocyclic derivative of 3-deazaadenosine (3-deaza-C-Ado), (RS)-3-adenine-9-yl-2-hydroxypropanoic acid [(RS)-AHPA] isobutyl ester, 3-deaza-C-Ado, 4-Amino-1-(2,3-dihydroxy-1-propyl)imidazo[4,5-c]pyridine, 1′-, 2′-, and 3-carbons of 3-deaza-C-Ado, 4-Amino-1-(4-hydroxy-1-butyl)imidazo[4,5-c]pyridine, 5˜-deoxy-S′-S-isobutyladenosin˜ (SIB A), (S)-9-(2,3-dihydroxypropyl)adenine, ribavirin, vidarabine, pyrazofurin, tubercidin, carbodine, (S)-9-(2,3-dihydroxypropyl)adenine [(S)-DHPA], 3-deaza-adenosine (DZA), 3-deaza-(+/−)aristeromycin (DZAri), 2′,3′-dideoxy-adenosine (ddAdo), 2′,3 ‘-dideoxy-3-deaza-adenosine (ddDZA), 2’,3′-dideoxy-3-deaza-(+/−) aristeromycin (ddDZAri), 3-deaza-5′-(+/−)noraristeromycin (DZNAri), 3-deazaneplanocin A (DZNep), and homodimer enzyme inhibitory antibodies to SAH inhibitors.

In some embodiments of the invention, the additional therapeutic agent includes at least one antiviral agent selected from Abacavir; Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Coviracil; Cytarabine Hydrochloride; Delavirdine Mesylate; Desciclovir; Didanosine; Disoxaril; Edoxudine; Emivirine; Emtricitabine; Enviradene; Enviroxime; Epivir; Famciclovir; Famotiite Hydrochloride; Fiacitabine; Fialuridine; Fosarilate; Foscarnet Sodium; Fosfonet Sodium; Ganciclovir; Ganciclovir Sodium; Idoxuridine; Indinavir; Kethoxal; Lamivudine; Lobucavir; Lodenosine; Lopinavir, Memotine Hydrochloride; Methisazone; Nelfinavir; Nevirapine;

Penciclovir; Pirodavir; Ribavirin; Rimantadine Hydrochloride; Saquinavir Mesylate; Ritonavir; Somantadine Hydrochloride; Sorivudine; Statolon; Stavudine; Tenofovir; Tilorone Hydrochloride; Trifluridine; Valacyclovir Hydrochloride; Vidarabine; Vidarabine Phosphate; Vidarabine Sodium Phosphate; Tipranavir, Viroxime; Zalcitabine; Zidovudine, Zinviroxime and Bromine Epiandrosterone. The compounds in U.S. Pat. No. 8,475,804, incorporated by reference in its entirety, may also be used. Any additional antiviral medication or medication for the treatment or coronaviruses may also be used as an additional therapeutic agent herein.

The additional therapeutic agent may also include vaccine candidates for coronaviruses which by utilizing the enhanced immune stimulation occurring in the patient can direct a viral specific immune clearance together with the generation of immune memory cells against the specific coronavirus presented in the vaccine.

In particular methods of the invention, a therapeutic agent, which that may be an additional therapeutic agent or may be administered alone as a separate embodiment of the invention, is a therapeutic or vaccine created by a neutralized coronavirus. In some case, a coronavirus may be neutralized by combining the virus prior to inoculation into a host or mammalian species with selected hydrophobic amino acid sequences. Such amino acid sequences may bind to the virus and decrease its infectivity and, in some cases, may allow for an increased immune activation against the coronavirus. Hydrophobic amino acid sequences include one or more of the following sequences, or any pharmaceutically acceptable salts, derivatives, metabolites, or prodrugs thereof:

Hydrophobic Amino Acid Sequences

DWYEDIIQAYREY

WIDSPFIWDNVMF

LTHWYAVDFNMWT

PWWYWEMRAFDAE

DWNIWDGWYREIY

RTHFEREFDDWFL

The compositions of embodiments of the invention can be administered to a patient in any of a wide range of routes. It is within the skill of the ordinary artisans to select a specific formulation and route of administration and then test suitability for use. Administration may be systemic or local. By way of example but not limitation, suitable routes include enteric, intravenous, parenteral, topical, oral, rectal, nasal or vaginal routes. Parenteral routes include subcutaneous, intramuscular, intravenous, intraperitoneal, intradermal and sublingual administration. Also, compositions may be implanted into a patient or injected using a drug delivery system which can slowly release the drug over an extended time frame utilizing drug slow release formulations already available in the art.

With regard to dosage and duration of treatment according to any aspect of the present invention, it is recognized that the ability of an artisan skilled in pharmaceutical administration of drugs to determine suitable dosages depending on many interrelated factors is well known, and skilled artisans are readily able to monitor patients to determine whether treatment should be started, continued, discontinued or resumed at any given time. For example, dosages of the compounds are suitably determined depending on the individual cases taking symptoms, age, weight and sex of the subject and the like into consideration. The amount of these currently prescription compounds to be incorporated into the pharmaceutical composition of the antiviral invention varies with dosage route, solubility of the compound, administration route, administration scheme and the like. An effective amount for a particular patient may vary depending on factors such as the condition being treated, the overall health of the patient and the method, route and dose of administration. The clinician using parameters known in the art makes determination of the appropriate dose. Generally, the dose begins with an amount somewhat less than the optimum dose and it is increased by small increments thereafter until the desired or optimum effect is achieved. Suitable dosages can be determined by further taking into account relevant disclosure in the known art.

In some embodiments of the invention, the amount of NSF antagonist compound is delivered to the patient is sufficient to achieve a plasma concentration of from about 3 to 10 μg/ml to about 15000 μg/ml of plasma, typically about 3 to about 50 μg/ml or about 5 to about 25 μg/ml. However, when liposomes targeted to coronavirus viral infected cells are used to administer the antagonists to the nuclear receptor family compounds, higher doses of 25 mg/ml or more may be used. The effective amount of the NSF antagonist compound is optionally administered in a dosage ranging between 10 μg/kg and about 20,000 μg/kg of body weight of the patient. Unit dosages for any of the conditions described in the disclosure will typically comprise about 1-1000 mg/day of an NSF receptor antagonists, and in particular, in a range of 5 to 500 mg/day, such as 400 mg/day with an optimal blood plasma concentration of 60 μM in the blood all day. In some embodiments, pediatric doses range from 0.001 to 100 mg/kg/day, with particular doses in the range of 3 mg/kg/day.

The treatments herein have been described for implementation using certain methods and apparatus for administering a components that include a nuclear steroid family (NSF) receptor antagonist, a calcium channel blocker, and a therapeutic agent, in an amount effective to treat or prevent the certain adverse conditions in mammals. Other forms of administration to a patient and additional combinations are included in the present invention.

For example, one or more of a nuclear steroid family (NSF) receptor antagonist, a calcium channel blocker, and a therapeutic agent, may be administered to a patient in an amount effective to treat or prevent the coronavirus infection via various modalities including, but not limited to: intravenous, subcutaneous injection, nebulizer, vape, dissolvable film strip, transdermal patch, transdermal cream (which may be combined with DMSO).

In addition, in some embodiments, a nuclear steroid family (NSF) receptor antagonist and a calcium channel blocker may be simultaneously or serially administered to a subject in the absence of the therapeutic agent.

In still other embodiments, a nuclear steroid family (NSF) receptor antagonist and a calcium channel blocker may be simultaneously or serially administered to a subject in combination with anti-inflammatory agents and/or compounds other than a therapeutic agent. For example, theaflavin, theaflavin-3-gallate and theaflavin-3′-gallate may be simultaneously or serially administered to the subject to further inhibit protease activity of the virus.

The invention is not limited to the embodiments described above but may be varied in both construction and detail within the scope of the claims.

We claim:

1. A method of treating or preventing a coronavirus infection in a subject in need thereof, comprising administering to the subject a nuclear steroid family (NSF) receptor antagonist and a calcium channel blocker, in an amount effective to treat or prevent the coronavirus infection.

2. The method of claim 1, wherein the NSF receptor antagonist comprises a mineralocortoid antagonist.

3. The method of claim 2, wherein the mineralocortoid antagonist comprises a spirolactone or a pharmaceutically acceptable salt, metabolite, or prodrug thereof.

4. The method of claim 3, wherein the mineralocortoid antagonist comprises at least one compound selected from spironolactone, spirorenone, dihydrospirenone, 1,2-dihydro-spirorenone, 1,2α-methylene-spirorenone, 7α-acetylthio-3-oxo-4,15-androstadiene-[17(β-1′)-spiro-5′]perhydrofuran-2′-one, 3-Oxo-7α-propionylthio-4,15-androstadiene-[17(β-1′)-spiro-5′]perhydrofuran-2′-one, 6β,7β-methylene-3-oxo-4,15-androstadiene-[17(β-1′)-spiro-5′]perhydrofuran-2′-one, 15α,16α-methylene-3-oxo-7α-propionylthio-4-androstene-[17(β-1′)-spiro-5′]perhydrofuran-2′-one, 6β,7β,15α,16α-dimethylene-3-oxo-4-androstene-[17(β-1′)-spiro-5′]perhydrofuran-2′-one, 7α-acetyithio-15α,16α-methylene-3-oxo-4-androstene-[17(β-1′)-spiro-5′]perhydrofuran-2′-one, 7α-acetylthio-15β,16β-methylene-3-oxo-4-androstene-[17(β-1′)-spiro-5′]perhydrofuran-2′-one, 15β,16β-methylene-3-oxo-7β-propionylthio-4-androstene-[17(β-1′)-spiro-5′]perhydrofuran-2′-one, 6β,7β,15β,16β-dimethylene-3-oxo-4-androstene-[17(β-1′)-spiro-5′]perhydrofuran-2′-one, eplerenone, iseplerenone, potassium canrenoate, canrenoate, canrenone, 7-α-thiospironolactone, 7α-thiomethylspironolactone, 6-β-hydroxy-7-α-thiospironolactone, 6-β-hydroxy-7-α-thiomethylspironolactone, and pharmaceutically acceptable salts, metabolites, and prodrugs thereof.

5. The method of claim 3, wherein the mineralocortoid antagonist comprises at least one compound selected from progesterone, gestodene, drospirenone, dimethisterone, ethinyl estradiol, ethisterone, 11β-hydroxyprogesterone, 17α-hydroxyprogesterone, 16α-methyl progesterone, hydroxyprogesterone caproate, medroxyprogesterone acetate, proligestone, metapristone, and mifepristone (11β-[p-(Dimethylamino)phenyl]-17β-hydroxy-17-(1 propynyl)estra-4,9-dien-3-one), and a pharmaceutically acceptable salt, metabolite, or prodrug thereof.

6. The method of claim 3, wherein the mineralocortoid antagonist is a compound having Formula B: 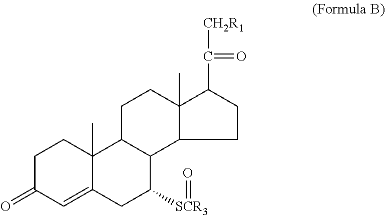
wherein R₁ is hydrogen, hydroxyl, a mineral acid ester such as sulfate, phosphate or nitrate group, or acyloxy-OR₂, the acyl group R₂ being derived from a carboxylic acid of the formula R₄OOH which may have up to 12 carbon atoms, and in which R₄ may be substituted or unsubstituted, saturated or unsaturated, straight chain or branched, alicyclic, aryl, heterocyclic or mixed and R₃ is methyl. In one embodiment, R₁ is hydroxyl or OR₂, or a pharmaceutically acceptable salt, metabolite, or prodrug thereof.

7. The method of claim 5, wherein the mineralocortoid antagonist comprises mifepristone or a pharmaceutically acceptable salt, metabolite, or prodrug thereof.

8. The method of claim 1, wherein the NSF receptor antagonist comprises at least one of the aglepristone, cyproterone, cyproterone acetate, casodex, besylate, bicalutamide, clomifene, femarelle, ormeloxifene, raloxifene, tamoxifen, toremifene, lasofoxifene, and ospemifene, nimodipine, afimoxifene, arzoxifene, fulvestrant, bazedoxifene, flutamide, nilutamide, and pharmaceutically acceptable salts, metabolites, and prodrugs thereof.

9. The method of claim 1, wherein the calcium channel blocker comprises at least one compound selected from amlodipine, aranidipine, azelnidipine, barnidipine, benidipine, cilnidipine, clevidipine, efonidipine, felodipine, isradipine, lacidipine, lercanidipine, manidipine, nicardipine, nifedipine, nilvadipine, nimodipine, nisoldipine, nitrendipine, pranidipine, and a pharmaceutically acceptable salt, metabolite, or prodrug thereof.

10. The method of claim 9, wherein the calcium channel blocker comprises amlodipine or a pharmaceutically acceptable salt, metabolite, or prodrug thereof.

11. The method of claim 1, wherein the NSF receptor antagonist and the calcium channel blocker are administered to the subject concurrently or sequentially.

12. The method of claim 11, wherein the NSF receptor antagonist is administered in a tablet or capsule, and the calcium channel blocker is administered in a tablet or capsule.

13. The method of claim 12, wherein the NSF receptor antagonist is mifepristone, and the calcium channel blocker is amlodipine.

14. The method of claim 1, wherein the nuclear steroid family (NSF) receptor antagonist, and the calcium channel blocker are administered to the subject in a single pharmaceutically acceptable composition.

15. The method of claim 1, further comprising administering to the subject at least one additional therapeutic agent.

16. The method of claim 15, wherein the at least one additional therapeutic agent is an antiviral agent selected from the group consisting of Carbocyclic 3-deazaadenosine (C-c³Ado), R- and S-isomers of 6′-C-neplanocin A analogues, carbocyclic analogues of adenosine, aristeromycin (carbocyclic adenosine), carbocyclic 3-deazaadenosine, neplanocin A (NepA), 3-deazaneplanocin A, 5′-nor derivatives of aristeromycin, carbocylic 3-deazaadenosine, 2-halo and 6-R-alkyl derivatives, neplanocin A, 9-(hydroxyalkenyl)purines (adenines and 3-deazaadenines), which analogues of neplanocin A, 3-deazaneplanocin A, bromine epiandrosterone, the 5′-nor derivatives of carbocyclic adenosine (C-Ado, aristeromycin), the 2-halo (i.e., 2-fluoro) and 6′-R-alkyl (i.e., 6′-R-methyl) derivatives of neplanocin A, 6′-C-methylneplanocin A (isomers I and II), 5′-noraristeromycin, (S)-9-(2,3-dihydroxypropyl)adenine, 5′-nor derivatives of carbocyclic adenosine (C-Ado, aristeromycin), 2-halo and 6′-R-alkyl derivatives of neplanocin A, 9-(hydroxyalkyl)-3-deazaadenines, which are analogues of the carbocyclic derivative of 3-deazaadenosine (3-deaza-C-Ado), (RS)-3-adenine-9-yl-2-hydroxypropanoic acid [(RS)-AHPA] isobutyl ester, 3-deaza-C-Ado, 4-Amino-1-(2,3-dihydroxy-1-propyl)imidazo[4,5-c]pyridine, 1′-, 2′-, and 3-carbons of 3-deaza-C-Ado, 4-Amino-1-(4-hydroxy-1-butyl)imidazo[4,5-c]pyridine, 5˜-deoxy-S′-S-isobutyladenosin˜ (SIB A), (S)-9-(2,3-dihydroxypropyl)adenine, ribavirin, vidarabine, pyrazofurin, tubercidin, carbodine, (S)-9-(2,3-dihydroxypropyl)adenine [(S)-DHPA], 3-deaza-adenosine (DZA), 3-deaza-(+/−)aristeromycin (DZAri), 2′,3′-dideoxy-adenosine (ddAdo), 2′,3′-dideoxy-3-deaza-adenosine (ddDZA), 2′,3′-dideoxy-3-deaza-(+/−) aristeromycin (ddDZAri), 3-deaza-5′-(+/−)noraristeromycin (DZNAri), 3-deazaneplanocin A (DZNep), and homodimer enzyme inhibitory antibodies to SAH inhibitors.

17. The method of claim 15, wherein the at least one additional therapeutic agent is an antiviral agent selected from the group consisting of Abacavir; Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Coviracil; Cytarabine Hydrochloride; Delavirdine Mesylate; Desciclovir; Didanosine; Disoxaril; Edoxudine; Emivirine; Emtricitabine; Enviradene; Enviroxime; Epivir; Famciclovir; Famotiite Hydrochloride; Fiacitabine; Fialuridine; Fosarilate; Foscarnet Sodium; Fosfonet Sodium; Ganciclovir; Ganciclovir Sodium; Idoxuridine; Indinavir; Kethoxal; Lamivudine; Lobucavir; Lodenosine; Lopinavir, Memotine Hydrochloride; Methisazone; Nelfinavir; Nevirapine; Penciclovir; Pirodavir; Ribavirin; Rimantadine Hydrochloride; Saquinavir Mesylate; Ritonavir; Somantadine Hydrochloride; Sorivudine; Statolon; Stavudine; Tenofovir; Tilorone Hydrochloride; Trifluridine; Valacyclovir Hydrochloride; Vidarabine; Vidarabine Phosphate; Vidarabine Sodium Phosphate; Tipranavir, Viroxime; Zalcitabine; Zidovudine, Zinviroxime and Bromine Epiandrosterone.

18. The method of claim 1, wherein at least one of the nuclear steroid family (NSF) receptor antagonist, and the calcium channel blocker, are administered in a formulation that comprises a liposome, carbohydrate, or cyclodextrin vehicle.

19. A pharmaceutical composition comprising:
a NSF receptor antagonist; a calcium channel blocker; and a pharmaceutically acceptable carrier.

20. The pharmaceutical composition of claim 19, wherein the NSF receptor antagonist comprises a mineralocortoid antagonist comprising one or more of: a spirolactone; a pharmaceutically acceptable salt; and a metabolite.
